(19) United States
(12) Patent Application Publication
(10) Pub. No.: US 2007/0088408 A1
(43) Pub. Date: Apr. 19, 2007

(54) Methods of reducing dermal melanocytes

(76) Inventor: Somnuk Amornsiripanitch, Nonthaburee (TH)

Correspondence Address: FISH & RICHARDSON PC, P.O. BOX 1022, MINNEAPOLIS, MN, 55440-1022, US

(21) Appl. No.: 11/580,353

(22) Filed: Oct. 13, 2006

Related U.S. Application Data
(60) Provisional application No. 60/726,303, filed on Oct. 13, 2005.

(51) Int. Cl. A61N 5/06; A61F 2/00; A61B 18/18

(52) U.S. Cl. 607/88; 606/9; 607/89; 607/101

(57) ABSTRACT

Disclosed herein are methods to reduce dermal melanocytes in a preselected dermal region of human skin afflicted with a disorder. The methods involve cooling an area of the skin above the preselected region and applying energy to the region to ameliorate any lesions of the disorder.

### CROSS-REFERENCE TO RELATED APPLICATIONS

This application claims the benefit of the filing date of U.S. Provisional Application No. 60/726,303, which was filed on Oct. 13, 2005. The contents of U.S. Application No. 60/726,303 are incorporated by reference as part of this application.

### TECHNICAL FIELD

This invention relates to methods applying energy to reduce dermal melanocytes.

### BACKGROUND

Dermal melanocytosis is characterized by, e.g., presence of ectopic melanocytes in the skin. It can be found in the disorders such as, e.g., Mongolian spot, blue nevus, nevus of Ota, nevus of Ito, and acquired bilateral nevus of Ota-like macules (ABNOM). The nature and the clinical significance of these disorders are different. The development of lasers has improved the treatment of these disorders.

### SUMMARY

The present invention is based, in part, upon the discovery that it is possible to reduce or even eradicate dermal melanocytes found in dermal melanocytosis disorders, e.g., acquired bilateral nevus of Ota-like macules (ABNOM), nevus of Ota, nevus of Ito, Mongolian spot, blue nevus, while at the same time preventing or minimizing damage to skin tissue surrounding dermal melanocytes afflicted with the disorder. In particular, dermal melanocytes, dermal regions containing blood vessels, and water content surrounding the dermal collagens and ground substances are targeted for heat injury, whereas the underlying dermal and overlaying dermal and epidermal regions are protected from thermal injury. The underlying dermal regions are protected from thermal injury because, by selection of appropriate parameters, it is possible to limit the penetration depth of the heating or energy applied to the region. Accordingly, by choice of appropriate parameters it is possible to heat skin tissue to a pre-selected depth thereby sparing the underlying tissue from thermal injury. The overlaying papillary dermal and epidermal regions are protected from thermal injury by appropriate surface cooling. Accordingly, by choice of appropriate heating and cooling parameters it is possible for the skilled artisan to focus thermal injury to a specific target zone within the dermis of the skin. The featured methods provide excellent results without unacceptable wounding the skin and produce fewer side effects such as, e.g., post-inflammatory hyperpigmentation and post-treatment hypopigmentation. In addition, repeat treatment can be performed faster, which can result in shorter time for the overall treatment.

In one aspect, the disclosure features a method of reducing or even eradicating dermal melanocytes, e.g., the number, size, density, and/or melanin content of dermal melanocytes, in a preselected dermal region of mammalian, e.g., human, skin, the preselected region having at least one lesion characteristic of the disorder disposed therein. The method includes the steps of: (a) cooling an area of the skin above the preselected dermal region; and (b) applying energy to the preselected dermal region in the absence of an exogenously provided energy absorbing material, in an amount sufficient to ameliorate the lesion. In the method, a temperature of the area of the skin above the preselected dermal region is below about 60 degrees Celsius before, during, or before and during the application of the energy.

Embodiments can include one or more of the following features.

The source of energy in step (b) can be selected from the group consisting of: laser light, incoherent lights, microwaves, ultrasound and radio frequency (RF) current. The source of energy in step (b) can be a laser light, e.g., a pulsed, scanned, or gated continuous wave (CW) laser. The source of energy, e.g., heating energy, in step (b) can be one or more beams of radiation, e.g., coherent or incoherent radiation, microwaves, ultrasound, or RF current. The energy, e.g., heat energy, in step (b) can originate from a radiation source, e.g., coherent radiation source. The source of energy in step (b) can be a laser or lasers that comprises a wavelength in the range from about 0.5 microns to about 1.8 microns. At least two types of energy, e.g., heating energy, can be applied in step (b). The two (or more) types of energy can be applied sequentially or contemporaneously. The source of energy in step (b) can be a laser or lasers that comprises at least two wavelengths that are applied sequentially. The source of energy, e.g., beam(s) of radiation, in step (b) can comprise at least two wavelengths in the range from about 0.5 microns to about 1.8 microns. The source of energy in step(b) can be a laser light that comprises a short wavelength and a longer wavelength, and wherein the short wavelength is applied before the longer wavelength. The source of energy in step (b) can be a laser light that comprises a short wavelength and a longer wavelength, and wherein the longer wavelength is applied before the short wavelength. The source of energy in step (b) can be a laser light that comprises at least three wavelengths that are applied sequentially. The source of energy in step (b) can be a laser light that comprises a short wavelength, a longer wavelength, and the longest wavelength, and wherein the longest wavelength is applied first, the longer wavelength is applied second, and the short wavelength is applied third. The source of energy in step (b) can be a laser light that comprises a short wavelength, a longer wavelength, and the longest wavelength, and wherein the short wavelength is applied first, the longer wavelength is applied second, and the longest wavelength is applied third. The source of energy in step (b) can be a laser light that comprises a short wavelength and a longer wavelength, and wherein the wavelengths are applied at random. The source of energy in step (b) can be a laser light that comprises at least two wavelengths that are applied contemporaneously.

The source of energy in step (b) can be a laser light that includes at least a short wavelength and a longer wavelength, and wherein the short wavelength is in the range from about 0.5 to about 1.0 microns, e.g., from about 0.6 to about 0.6 microns, and/or the longer wavelength is in the range from about 1.0 to about 1.8 microns, e.g., from about 1.3 to about 1.5 microns, or from about 1.0 to about 1.1 microns. The short wavelength can comprise a fluence in the range from about 2 joules to about 25 joules per square centimeter and/or a duration from about 0.45 milliseconds to about 40 milliseconds, e.g., from about 0.45 milliseconds to about 25 milliseconds. The longer wavelength can comprise a fluence in the range from about 4 joules to about 150 joules per square centimeter, e.g., from about 6 joules to about 150 joules per square centimeter and/or a duration from about 0.25 milliseconds to about 300 milliseconds.

Step (a) of the present methods can occur prior to and/or after and/or contemporaneously with step (b). Cooling an area of the skin in step (a) can be achieved by many different techniques known in the art, e.g., by blowing a stream of cold air or gas onto the target area of the skin, by applying a cold liquid onto the target area, by conductive cooling using a cold contact surface applied to the target area, or by evaporative cooling using a low-boiling-point liquid applied to the target area. In a preferred embodiment, cooling is achieved using evaporative cooling technologies by means of, e.g., a commercially available dynamic cooling device (DCD).

The disorder of the present methods can be, e.g., Nevus of Ota or Nevus of Ito. The disorder can be Nevus of Ota. At least one lesion of the disorder or the present methods can have hypermelanotic color, and applying energy in step (b) can lighten the hypermelanotic color of at least one lesion and/or reduce density of the lesions disposed within the preselected region.

The disorder of the present methods can be a freckle of Hori and the source of energy applied in step (b) can be a laser or lasers that comprises a short wavelength and a longer wavelength, and wherein the wavelengths are applied sequentially. The short wavelength can be applied prior to the application of the longer wavelength. The short wavelength can be applied contemporaneously with the longer wavelength. The short wavelength can be from about 0.5 to about 0.6 microns and/or have a fluence in the range of about 2 joules to about 25 joules per square centimeter. The longer wavelength can be from about 1.0 to about 1.5 microns and/or have a fluence in the range of about 4 joules to about 150 joules per square centimeter. The short wavelength light can have a duration from about 0.45 milliseconds to about 40 milliseconds and/or the longer wavelength can have a duration from about 0.25 milliseconds to about 300 milliseconds.

The present methods can be repeated weekly, biweekly or monthly until the lesion(s) is reduced and/or disappears. The present methods can be performed along with other methods, e.g., along with treatments with Q-switch (QS) lasers, e.g., QS ruby, QS alexandrite, and/or QS Nd/YAG, carried out to reduce the problems of dermal melanocytosis, e.g., freckle of Hori, Nevus of Ito, or Nevus of Ota. The present methods can be carried out prior to, between, or after QS laser treatment methods.

The disorder of the present methods can be a freckle of Hori and the source of the energy in step (b) can be a laser or lasers comprising a short wavelength, a longer wavelength and a longest wavelength, wherein the wavelengths are applied sequentially. The short wavelength can be applied, prior to the application of the longer and the longest wavelengths. The longest wavelength can be applied first, the longer wavelength can be applied second, and the short wavelength can be applied third. The sequential application of more than two wavelengths can be at random. The three wavelengths can be applied contemporaneously. The short wavelength can be from about 0.5 to about 0.6 microns and/or have a fluence in the range from about 2 joules to about 25 joules per square centimeter. The longer wavelength can be from about 0.6 to about 1.1 microns and/or have a fluence in the range from about 4 joules to about 150 joules per square centimeter. The longest wavelength can be from about 1.1 to about 1.5 microns and/or have a fluence in the range from about 4 joules to about 20 joules per square centimeter. The duration of the short wavelength of the laser light can be from about 0.45 milliseconds to about 100 milliseconds. The duration of the long and/or longest wavelength can be from about 0.25 milliseconds to about 300 milliseconds. The methods wherein the source of energy in step (b) is a laser or lasers with least three wavelengths and wherein the disorder is a freckle(s) of Hori can be repeated weekly, biweekly or monthly until the lesion(s) is reduced or disappears.

The disorder of the present methods can be a freckle of Hori and the source of the energy in step (b) can be incoherent lights or intense pulse lights composed of shorter and longer wavelengths. The incoherent or intense pulse lights can comprise at least two wavelengths of the ranges, fluence, and duration described herein, e.g., analogous to laser light wavelengths described herein. The incoherent or intense pulse lights can be used in an analogous fashion to the laser light uses described herein.

The disorder of the present methods can be a freckle of Hori. At least one lesion of the disorder of the present methods can have hypermelanotic color, and applying energy in step (b) can lighten the hypermelanotic color of at least one lesion and/or reduce density of the lesions disposed within the preselected region.

In another aspect, the disclosure features a method of reducing or even eradicating dermal melanocytes, e.g., number, size, density and/or melanin content of dermal melanocytes, in a preselected dermal region of mammalian, e.g., human, skin, the preselected region having at least one lesion characteristic of the disorder disposed therein. The method includes the steps of: (a) cooling an area of the skin above the preselected dermal region; and (b) applying energy to the preselected dermal region in the presence of an exogenously provided energy absorbing material, in an amount sufficient to ameliorate the lesion. In the method, a temperature of the area of the skin above the preselected dermal region is below about 60 degrees Celsius before, during, or before and during the application of the energy.

Embodiments can include the features described above, as well as the following.

The source of the energy in step (b) can be radiation, and the exogenously provided energy absorbing material can be a radiation absorbing material, e.g., a chromophore photoexcited by the radiation. The energy absorbing material, e.g., a radiation absorbing material, can be administered systematically to the mammalian, e.g., human, skin, or applied topically to a preselected region of the skin prior to application of energy, e.g., radiation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the disclosure will be apparent from the following detailed description, and from the claims.

### DESCRIPTION OF DRAWINGS

FIG. 1A is a graph depicting absorption coefficient of water by the light in the related wavelength. Water begins to be absorbed at about 800 nm and the absorption decreases at about 1000 to 1100 nm. The absorption then increases sharply until it reaches the wavelength of about 1450 nm. The absorption then decreases to the wavelength of about 1700 nm and again increases to the highest peak at about 1940 nm.

FIG. 1B is a graph depicting absorption spectra of four skin chromophores: oxyhemoglobin, deoxyhemoglobin, melanin and water. Each of the four curves corresponds to the absorption spectrum of a different chromophore. The absorption coefficient of water is the enhanced in detail in the graph of FIG. 1A. The absorption coefficient of both hemoglobins shows some differences around 500-800 nm, after which the absorption is similar. Melanin absorption shows a regression from about 500 nm to about 1100 nm.

FIG. 1C is a graph depicting the depth of light penetration of human skin at various wavelengths. Light at different wavelengths, from about 400 nm to about 2000 nm, penetrates skin differently. At about 500-600 nm, the light can penetrate down to about 500 microns (0.5 mm). Light in the range of about 630 nm to about 980 nm can penetrate the skin to the level of more than about 1500 microns (1.5 mm). Light in the range of about 1000-1100 nm can penetrate down almost 3500 microns (3.5 mm). Light at 1450 nm can penetrate to a low level, similar to light with 600 nm wavelength.

FIG. 2A is a drawing depicting a cross-sectional area of the facial skin with dermal melanocytes, as generally seen in, e.g., acquired bilateral nevus of Ota-like macules (ABNOM). Superficial blood vessels (2a and 2b) and deep blood vessels (2c) are seen under normal epidermis (1). A sebaceous gland (3) attached to a hair follicle (4) is generally observed at the depth of about 500-700 microns in the dermal layer. Collagen bundles can be seen in both superficial dermis (5a) and deep dermis (5b). Subcutaneous layer filled with fat cells is at the bottom (6). Dermal melanocytes (7a, 7b and 7c) reside throughout the superficial dermis. The melanocytes can be located adjacent to blood vessels (7a) and along the collagen bundles in superficial dermis (7b) and deep dermis (7c).

FIG. 2B is a drawing depicting a cross-sectional area of skin being treated with a laser light (9a). The laser light can have a wavelength of, e.g., about 500-1000 nm, e.g., 500-600 nm. The drawing shows light penetrating down to about 500 microns at the level of the sebaceous gland (3). The light is absorbed by hemoglobin in superficial blood vessels (2a) and melanin in dermal melanocytes (7a, 7b). A blood vessel outside the radiation field (2b), a blood vessel at the deeper level (2c), and a deep dermal melanocyte (7c) are not affected by the radiation. A dermal melanocyte residing in superficial dermis (7b), but not near the blood vessels is mildly affected by the radiation. An area of edema (8a) caused by immediate inflammation after the radiation occurs at the superficial dermis.

FIG. 2C is a drawing depicting a cross-sectional area of skin being treated with a laser light (9b). The drawing depicts the area that has been treated with a laser as shown in FIG. 2B. Here, laser light (9b) has a higher wavelength of, e.g., about 1000-1800 nm, e.g., about 1000-1100 nm. The longer wavelength can penetrate down more than 3000 microns. It is absorbed by hemoglobin in both the superficial blood vessels (2a) and deep blood vessels (2c). Dermal melanin and dermal melanocytes in both superficial level (7a, 7b) and deep level (7c) absorb the light. Areas of edema deepen (8b).

FIG. 2D is a drawing depicting a cross-sectional area of skin that has been treated with several laser radiations as shown in FIG. 2B and FIG. 2C. The edema fills the dermal layer of the skin. The edema can be seen as a swollen skin (1b) compared with the normal adjacent skin (1a). The melanin dust is shown as 7a, 7b, and 7c.

FIG. 3 is a drawing depicting a cross-sectional area of the facial skin with dermal melanocytes as generally observed in, e.g., Nevus of Ota. Under normal epidermis (1), there are superficial blood vessels (2a and 2b) and deep blood vessels (2c). A sebaceous gland (3) attached to a hair follicle (4) is generally observed at the depth of about 500-700 microns in the dermal layer. Collagen bundles can be seen in both superficial dermis (5a) and deep dermis (5b). Subcutaneous layer filled with fat cells is at the bottom (6). Dermal melanocytes (7a, 7b and 7c) reside throughout the superficial dermis. They can be located adjacent to blood vessels (7a) and along the collagen bundles in the superficial dermis (7b) and deep dermis (7c). Without being limited to a particular theory, the condition generally demonstrates a deeper depth of involvement, more clusters of dermal melanocytes, about, e.g., 4.5 times more cells and about, e.g., 2.7 times more pigment when compared with ABNOM illustrated in FIG. 2A.

Like reference symbols in the various drawings indicate like elements.

### DETAILED DESCRIPTION

Increase in dermal melanocytes in human dermal skin is called dermal melanocytosis. It is part of disorders such as, e.g., Mongolian spot, blue nevus, nevus of Ota, nevus of Ito, and acquired bilateral nevus of Ota-like macules (ABNOM, also known as freckle of Hori). The nature and the clinical significance of these disorders are different. The methods featured herein include reducing dermal melanocytes, e.g., reducing size, density, number, or melanin content of dermal melanocytes, in a preselected dermal region of mammalian, e.g., human skin affected by any of these disorders by, inter alia, cooling an area of the skin above the preselected dermal region, and applying energy to the region. The energy can be applied by, e.g., a laser or lasers with varying wavelengths. When more than one wavelength of laser light is applied, the various wavelengths can be applied in various sequences or randomly or contemporaneously. The wavelengths of, e.g., laser light, are chosen and applied to selectively target specific areas and depths of the skin.

Disorders that Can Be Treated

Any dermal melanocytosis disorder can be treated by the featured methods. Such disorders include, e.g., Mongolian spot, blue nevus, nevus of Ota, nevus of Ito, and ABNOM.

Mongolian spot occurs during the newborn period at the lumbosacral and buttock areas and usually disappears spontaneously in a few years. Blue nevus can occur in a single lesion predominantly on hands and feet and does not present with progressive spreading pattern.

Nevus of Ota and Nevus of Ito share similarity of skin manifestation but are generally localized different areas of the body. Nevus of Ota affects facial skin supplied by the ophthalmic and maxillary divisions of the trigeminal nerve. It can also involve areas of the eyes such as the sclera, cornea, iris and retina, uveal tract as well as nasopharynx, auricular mucosa, tympanic membrane and dura. The hard palate inside the mouth also can be involved. In rare situations the disorder is reported with an ipsilateral sensineural deaffiess or glaucoma of the eye. Melanomas can develop in the skin, eye and brain of these patients. Nevus of Ito manifests at the skin enervated by the posterior supraclavicular and lateral brachial cutaneous nerves.

Acquired bilateral nevus of Ota-like macules (ABNOM) was described by Hori et al. in J. Am. Acad. Dermatology, 1984 June; 10(6): 961-4. It is known by several terms such as nevus of Hori, Hori's macules, freckles of Hori and acquired symmetrical dermal melanocytosis (ASDM). It manifests as blue-brown macules of the face occurring on both sides of the forehead, temple, eyelids, malar area, alae of the nose, and root of the nose. It is often is observed in middle-aged Asian women. The macules differ clinically from nevus of Ota, but share similar aesthetic problems since both disorders occur on the face and do not spontaneously disappear. The featured methods can reduce the aesthetic problems associated with these and other disorders.

Nevus of Ota can occur both at younger age and older age. In contrast ABNOM occurs more frequently at the older age. ABNOM is commonly known to develop after 15 years of age (mean age 36) in about 94% of the cases. Therefore correcting the aesthetic problems of Nevus of Ota can be carried out earlier than correcting problems of ABNOM, which is generally treated at middle age when the lesions are more progressive.

Details of the Methods

The featured methods are comprised of at least two steps.

In the first step, an exposed surface of a preselected region of mammalian skin having at least one lesion characteristic of a dermal melanocytosis disorder is cooled. The cooling step can be carried out with the cooling device that uses cold air, cold compress or compression using, e.g., a gel, cold contact or cold pack, or the delay cooling device (DCD). In the preferred embodiment, DCD is used in the laser system of Vbeam®, GentleYAG®, Smoothbeam™ (the product of Candela Corporation, 530 Boston Post Road, Wayland, Mass. 01778 USA). The preferred cooling system, DCD, allows a user to select both the duration of the spray cooling time in milliseconds and the delay of the spray cooling to the start of the radiation of laser light in another setting of milliseconds. This precise setting allows a user to record and review the results of each treatment to optimize the results and to minimize the epidermal damage.

In the second step, heating energy, for example, laser radiation, is applied to the preselected region in an amount and for a time sufficient to induce thermal damage to a portion of the skin containing dermal melanocytes to thereby reduce or eliminate or alter the structure of the dermal melanocytes. In this second step, at least two types of laser energy are selected to heat the area.

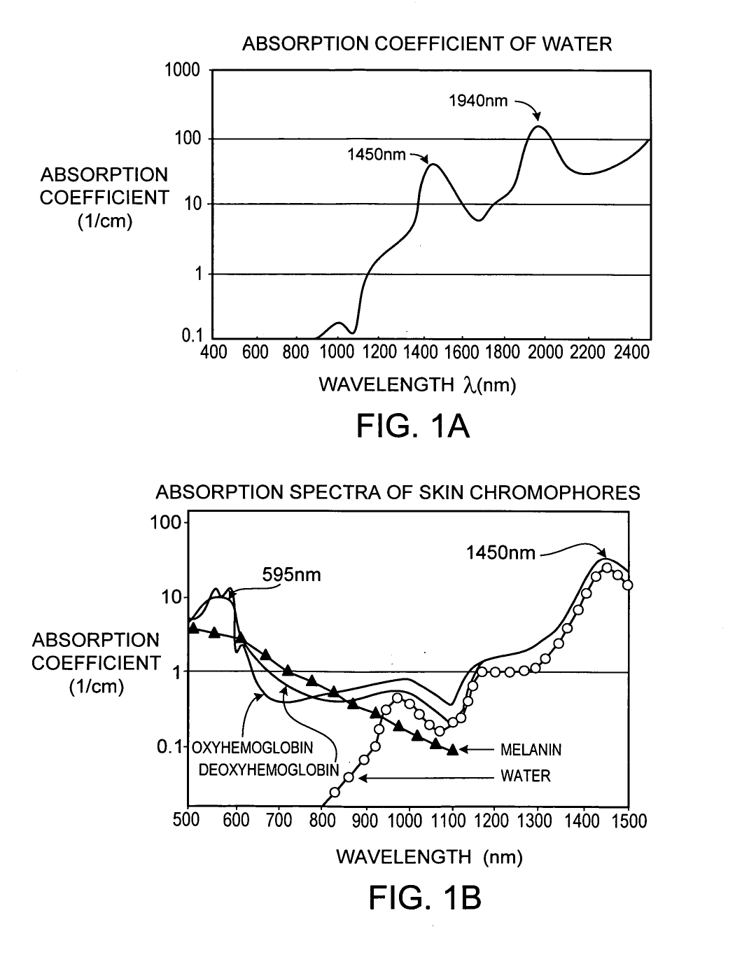

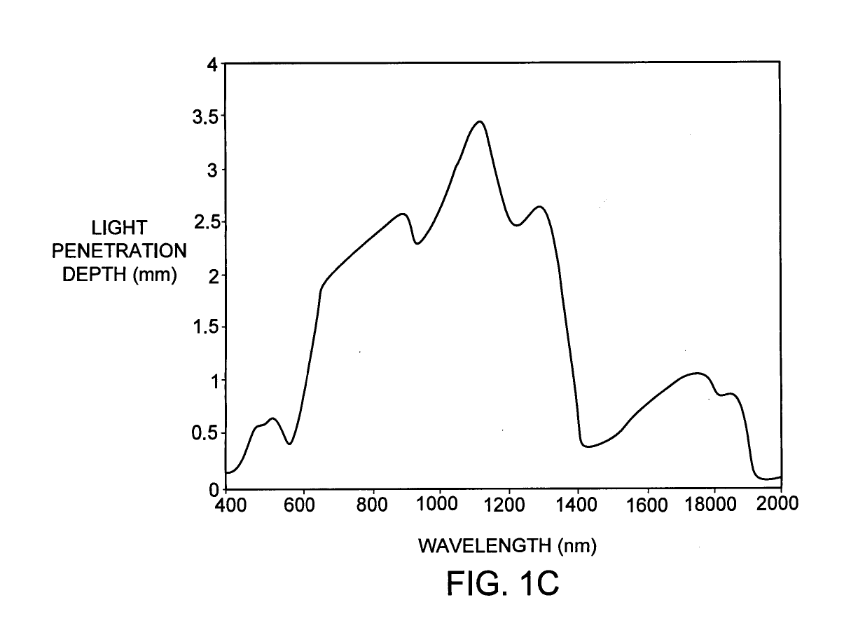

The short wavelength is selected to heat the superficial blood vessels carrying, e.g., nutrients to nourish the dermal melanocytes. The short wavelength also has the specific photothermolysis wavelength to target the melanin pigment in the dermal melanocytes. This short wavelength laser can be selected from the laser systems that produce the wavelength in the range well-absorbed by oxy- and deoxygenated hemoglobin in dermal blood vessels, as well as in the range well-absorbed by dermal melanin. As demonstrated in FIG. 1A and FIG. 1B, the preferred selected wavelength is from about 585 to about 600 nm. The preferred energy of the laser light of the preferred short wavelength laser systems is in the range of about 2-12 joules per centimeter square and the pulse duration is between about 1.5 milliseconds to about 6 milliseconds. The wavelength that is shorter than the preferred wavelength, for example a wavelength of about 530-540 nm, generally cannot penetrate deep enough and can harm the epidermis more than the preferred wavelength. There may be applications, however, when a shorter wavelength can be useful. In such applications, for example, the wavelength of 532 nm, the KTP/532 nm frequency-doubled neodymium: YAG laser can be used. The wavelength that is longer than the preferred wavelength of about 585-600 nm is absorbed less by hemoglobin than by dermal melanin. Thus, using a laser with the wavelength longer than about 595 nm will affect blood vessels to a lesser extent than dermal melanocytes. There may be applications, however, when a longer wavelength can be useful. If the longer wavelength is needed, it can be selected from, e.g., the wavelength of about 670-810 nm (for example, the Alexandrite laser at 755 nm and sapphire window cooled super-long-pulse 810 nm diode). The optimal energy of 700-810 nm laser system is about 10-50 joules per centimeter square with the pulse duration in the range of 1.5-10 milliseconds. This disclosed methods include all the wavelengths described herein but the preferred short wavelength is about 585-600 nm.

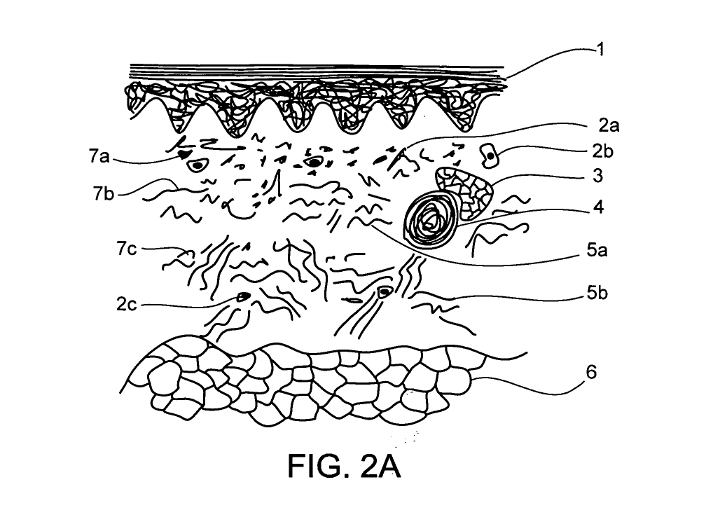

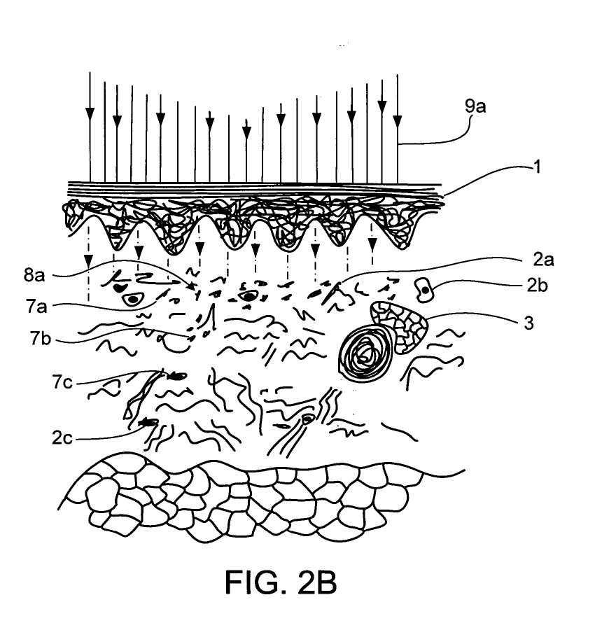

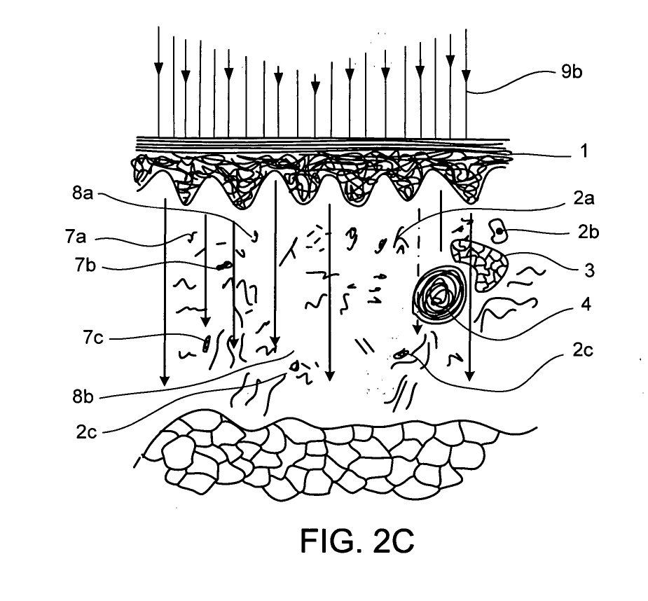

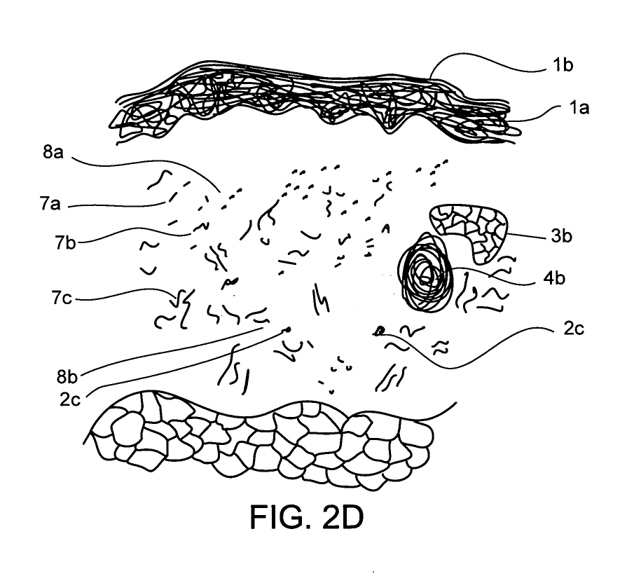

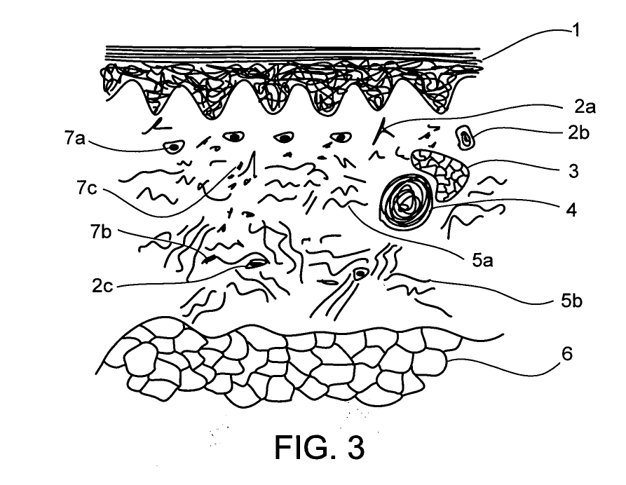

Referring to FIG. 2A, a cross-sectional area of facial skin is shown. Dermal melanocytes 7a, 7b, and 7c in acquired bilateral nevus of Ota-like macules (ABNOM) are generally scattered in the areas both close to and far from dermal blood vessels. The cells are located mostly in the superficial dermis at the depth of less than 1000 microns or 1 mm. FIG. 2A also shows the epidermis 1, and the superficial blood vessels 2a and 2b that are seen in longitudinal and cross-sectional views. Larger blood vessels 2c residing in the deeper levels of dermis are also present, along with a sebaceous gland 3, which is found generally in human facial skin, and a hair follicle 4 shown in cross-section. Both the sebaceous gland 3 and the hair follicle 4 structures are generally at the depth level of less than 1000 microns or 1 mm. Collagen bundles 5a and 5b residing in the superficial and deep dermis are shown. A subcutaneous layer 6 is composed of many fat cells. Dermal melanocytes 7a, 7b and 7c found in ABNOM can reside around superficial blood vessels, as illustrated by melanocyte 7a. Sometimes the melanocytes reside between collagen bundles both at superficial level (7b) and at deeper level (7c). However, as shown in FIG. 3, the dermal melanocytes (7a, 7b and 7c) in nevus of Ota normally cluster more densely, with about 4.5 times more cells and about 2.7 times more melanin pigments than those found in dermal melanocytes of ABNOM. The depth of dermal melanocytes in nevus of Ota can reach the depth of about 1.6 mm. All the other numbers in FIG. 3 illustrate normal epidermal and dermal structures analogous to those shown in FIG. 2A.

Referring to FIG. 2B, the specific energy from the selected short wavelength laser 9a will cause the damage of dermal melanocytes (7a) and blood vessels (2a) resulting in local accumulation of lymphatic fluid and water in the treated area (8a) to the average depth of less than about 1 mm or 1000 microns (at the mean of about 500 microns), close to the depth level of sebaceous gland (3). Blood vessels outside the irradiated field (2b), blood vessels residing too deeply in dermis (2c) will not be damaged. The very small blood vessels with diameter of less than 30 microns, e.g., 5-20 microns, will be mildly damaged and survive to nourish the irradiated tissue further. Dermal melanocytes residing too deeply (7c) and not closely to superficial blood vessels (7b) will survive from the damage. However, the destruction of some blood vessels will cause leakage of deoxyhemoglobin into the treated area, which will result in the increase of chromophores in the hidden dermal melanocytes at both superficial and deep dermis. Further applications of laser lights with more specific absorption will damage the leftover dermal melanocytes and supporting structures.

As demonstrated in FIG. 2C, the longer wavelength laser (9b) is selected to target and heat the deeper blood vessels (2c) and melanin both outside and inside dermal melanocytes (7b and 7c) that the short wavelength (9a in FIG. 2B) cannot reach or cannot completely destroy. The lymphatic fluid and water (8a) previously induced by the short wavelength in the treated area can also be affected further by the longer wavelength, resulting in further damage of the superficial blood vessels (2a) and dermal melanocytes (7a) in FIG. 2B. This longer wavelength laser is selected from the wavelength above about 1.0 microns or 1000 nanometers. The preferred infrared laser is selected from the wavelength between about 1.0 microns and about 1.1 microns. FIG. 1B shows that this preferred wavelength will be absorbed by both melanin and hemoglobin, and also by the water in the dermal area in the best suitable absorption coefficient to provide the best outcome of the treatment described herein. FIG. 1C also shows that this preferred wavelength can penetrate deeper into the skin. Therefore, referring to FIG. 2C, the radiation of this infrared laser will add the damage to leftover blood vessels (2c), both the larger and the smaller vessels, both connected and not connected to the damaged vessels, containing both oxy and deoxygenated hemoglobin. The radiation will also cause some damage to dermal tissue (8a and 8b) by transferring the energy to the leakage of deoxyhemoglobin, water and lymphatic fluid in the irradiated area.

The preferred energy of the laser light of the preferred infrared wavelength laser systems is in the range of about 1080 joules per centimeter square and the pulse duration is between about 0.25 milliseconds to 300 milliseconds. The wavelength from about 600-800 nm has a high coefficient of absorption by melanin that is too high when compared with hemoglobin, which will harm protected epidermal layer containing high melanin content as seen in, e.g., Asian skin, which is predominantly affected by dermal melanocytosis. In QS laser treatments for dermal melanocytosis, the use of QS Ruby laser (690 nm), QS Alexandrite laser (755 nm) and QS Nd/YAG laser (1064 nm) can produce a similar outcome. Therefore, it is possible to use long pulse laser with either one of 690 nm, 755 nm and 1064 nm in combination with the short wavelength (about 500-600 nm) in the present invention. As seen in FIG. 1B, the 800-920 nm light demonstrates the absorption coefficient of hemoglobin and melanin relatively similar to the one seen with 500-600 nm, but not similar to the absorption coefficient of water. Therefore, the use of this wavelength does not create any more benefit. The use of 800-920 nm laser light is an alternative to the use of 500-600 nm laser light with lower effectiveness, since the energy will be lost into the dermal water. The wavelength of far visible light and short infrared light shorter than the preferred infrared light, such as the wavelength of light less than 1.0 microns (e.g., about 920-980 nm) can also be used since it will be absorbed by hemoglobin, water and melanin, but the coefficient of the absorption is not equal to the absorption of the preferred wavelength, which may result into the shallower the penetration. On the other hand, the wavelength longer than the preferred range of about 1000-1100 nm will not be absorbed by melanin but will be absorbed more by water and deoxyhemoglobin and will not penetrate deeply enough in the skin as shown in FIG. 1C. If it is selected, this longer infrared can be selected from the wavelength of about 1450 nm since its depth is equal to the depth penetration of the 500-600 nm light. The usage of this 1450 nm wavelength will add to the destruction of the dermal vascular and dermal edema areas created by 500-600 nm radiation. Therefore, in the cases with deeper lesions a better outcome can result from the usage of the second wavelength of about 1000-1100 microns. The cases with shallow lesions can be given the second wavelength of about 1000-1100 microns laser and/or 1450 nm laser. The addition of 1450 nm radiation to the 1000-1100 microns is beneficial since the water absorption coefficient between them has almost a 200-time difference.

The disclosed invention, therefore, claims all the wavelengths mentioned in this description to reduce or even eradicate dermal melanocytes. During the second step, the preferred energy is selected from laser lights with the first wavelength chosen from the light with wavelength of about 585-600 nm, the second wavelength chosen from the wavelength of about 1064 mn or 1079 nm, and the third wavelength chosen from the wavelength of about 1450 nm. Use of fewer or more than three types of wavelengths is encompassed by the methods. Although the preferred embodiments of the wavelength are provided as an example, the usage of other wavelengths described herein is also encompassed by the present invention. The invention encompasses uses of the described wavelengths in the same order as described, in the reverse order, in random order, or contemporaneously in any session of the treatment.

Referring to FIG. 2D, the result of the combined treatments with the optimum range of energy will result into the specific swelling (1a) or mild purpura of all the lesions in the treated area with normal skin (1b). The swelling will last from hours to a few days, generally without the need of wound care.

All the steps of the featured methods can be repeated weekly, biweekly or monthly until the lesions are reduced or even eradicated.

Dermal melanocytosis, such as Nevus of Ota and ABNOM can be treated with the usage of QS lasers. QS lasers such as QS Ruby, QS Alexandrite and QS Nd/YAG can be used with the methods described herein. Usages of QS laser treatments prior to, in between, or after the usage of the present invention to hasten the outcome of the treatment do not depart from the spirit of the invention nor the scope of the claims.

A number of embodiments of the invention have been described. Nevertheless, it will be understood that various modifications may be made without departing from the spirit and scope of the invention. For example, the disclosed methods can be used in conjunction with application by other laser machines generating similar or close wavelengths as alternatives. Some alternative lasers include: products of Cynosure (Cynosure, Inc-5 Carlisle Road, Westford, Mass. 01886, U.S.A.), VStar and PhotoGenica V with the wavelength of 585 nm and 595 nm, Acclaim 7000 Nd/YAG and SmartEpil II Nd/YAG with the wavelength of 1064 nm; the products of CoolTouch™ (9085 Foothills Boulevard, Roseville, Calif. 95747, U.S.A.), such as CoolTouchCT3™ with the wavelength of 1320 nm and CoolTouchVARIA™ with the wavelength of 1064 nm; the products of Laserscopee; the products of Fotona®, etc. U.S. Pat. No. 6,613,042 describes a laser system that can generate five laser wavelengths (540 nm, 598 nm, 670 nm, 1079 nm, and 1341 nm) in one machine. The machine of this patent can also be used in the methods of the present invention to reduce or even eradicate dermal melanocytes. Depending on the more precise energy under the fixed wavelengths, duration of radiation, and the sequence of order of the multiple lights, an even better laser system or machine can be built to specifically help reduce or eradicate dermal melanocytes with few or no side effects. Such new system will have a lower cost and require fewer laser machines reducing the amount of space that they require.

Thus, the new laser system disclosed herein can function alone or in addition to the QS laser systems known to treat dermal melanocytosis. The conventional laser system that can affect blood vessels superficially, such as 532 and 585-595 nm, and the infrared laser that can affect deeper blood vessel, such as Nd/YAG (1064 nm), can be used in the methods and new laser system disclosed herein. Other infrared lasers that can act on water in the dermal layer, such as 1320 and 450 nm lasers, are also encompassed by the present methods. The novel methods of skin cooling currently used in noninvasive laser can be used in treating dermal disorders without damage to epidermis as described herein.

Both flash lamp pulsed dye laser and long pulsed tunable dye laser with the wavelength between 585-595 nm are encompassed by the present invention, as is infrared laser with wavelength between 1000-1500 nm.

The attempt to use multiple laser systems, such as the combination of the pulsed dye laser and infrared laser, to remove or reduce dermal melanocytes, therefore, is a novel method that can eradicate or reduce dermal melanocytosis with minimal side effects. The featured methods will especially benefit many Asian people. The new laser system built on the ideas of the present invention will cost less and occupy less space than the requirement of the combination of multiple laser machines to achieve the results presented in this invention.

### EXAMPLE

The following example of the use of the featured method should not be used to limit the scope of the claims.

1. A lesion of ABNOM on skin is identified.

2. Parameter settings on Candela, Vbeam (595 nm laser system, the product of Candela Corporation), are set at 5-6 joules per centimeter square with the pulse duration of 1.5-3 ms.

3. DC D setting is at 20-30 ms with 10-20 ms delay.

4. The prepared laser light with the wavelength of 595 nm is used to irradiate the selected lesion.

5. Parameter settings on Candela, GentleYAG (1064 nm laser system, the product of Candela), are set at 40-50 joules per centimeter square with the pulse duration of 3-5 ms.

6. DC D setting of GentleYAG is set at 20-30 ms with 10-20 delay.

7. The prepared laser light with the wavelength of 1064 nm is used to irradiate the lesion previously radiated by Vbeam.

8. In the alternative, during step 5 and/or 6, Smoothbeam (1450 nm laser system, the product of Candela Corporation) is used instead of GentleYAG. Parameters are set at 12-13 joules per centimeter square with the pulse duration total of 250 ms and the DCD is set at 20-30 ms.

9. The prepared laser light with the wavelength of 1450 is used to irradiate the lesion previously radiated by Vbeam or by both Vbeam pulsed GentleYAG.

10. The end point of each treatment is the specific swelling with or without mild purpura of the irradiated lesion, which will last a few hours to a few days.

11. The optimal parameters and the sequence of all the three laser systems are recorded to use as guideline for the next session.

12. The sequence of order in each session can be as the following:
The preferred embodiment has the following order:
Vbeam, GentleYAG, or
Vbeam, Smoothbeam, or
Vbeam, GentleYAG, Smoothbeam, or
Vbeam, Smoothbeam, GentleYAG
In another embodiment, the order can be:
Gentle YAG, Vbeam, or
Smoothbeam, Vbeam, or
Gentle YAG, Vbeam, Smoothbeam, or
Smoothbeam, Vbeam, GentleYAG, or
Smoothbeam, GentleYAG, Vbeam, or
GentleYAG, Smoothbeam, Vbeam

13. In subsequent sessions, order different from that of 12 can be used, depending on the characteristics of the lesions.

A number of embodiments of the invention have been described. Nevertheless, it will be understood that various modifications may be made without departing from the spirit and scope of the invention. Accordingly, other embodiments are within the scope of the following claims.

What is claimed is:

1. A method of reducing dermal melanocytes in a preselected dermal region of human skin, the preselected region having at least one lesion characteristic of the disorder disposed therein, the method comprising the steps of:
(a) cooling an area of the skin above the preselected dermal region; and
(b) applying energy to the preselected dermal region in the absence of an exogenously provided energy absorbing material, in an amount sufficient to ameliorate the lesion,
wherein a temperature of the area of the skin above the preselected dermal region is below about 60 degrees Celsius before, during, or before and during the application of the energy.

2. The method of claim 1, wherein in step (b) the source of energy is selected from the group consisting of: laser light, incoherent lights, microwaves, ultrasound and RF current.

3. The method of claim 1 wherein in step (b) the energy is provided by laser light.

4. The method of claim 3, wherein the laser light comprises a wavelength in the range from about 0.5 microns to about 1.8 microns.

5. The method of claim 3, wherein the laser light comprises at least two wavelengths that are applied sequentially.

6. The method of claim 5, wherein the laser light comprises a short wavelength and a longer wavelength, and wherein the short wavelength is applied before the longer wavelength.

7. The method of claim 5, wherein the laser light comprises a short wavelength and a longer wavelength, and wherein the longer wavelength is applied before the short wavelength.

8. The method claim 3, wherein the laser light comprises at least three wavelengths that are applied sequentially.

9. The method of claim 8, wherein the laser light comprises a short wavelength, a longer wavelength, and the longest wavelength, and wherein the longest wavelength is applied first, the longer wavelength is applied second, and the short wavelength is applied third.

10. The method of claim 8, wherein the laser light comprises a short wavelength, a longer wavelength, and the longest wavelength, and wherein the short wavelength is applied first, the longer wavelength is applied second, and the longest wavelength is applied third.

11. The method of claim 5, wherein the laser light comprises a short wavelength and a longer wavelength, and wherein the wavelengths are applied at random.

12. The method of claim 3, wherein the laser light comprises at least two wavelengths that are applied contemporaneously.

13. The method of claim 6, wherein the short wavelength of laser light is in the range from about 0.5 to about 1.0 microns.

14. The method of claim 6, wherein the longer wavelength of laser light is in the range from about 1.0 to about 1.8 microns.

15. The method of claim 13, wherein the short wavelength of laser light comprises a fluence in the range from about 2 joules to about 25 joules per square centimeter.

16. The method of claim 14, wherein the longer wavelength of laser light comprises a fluence in the range from about 4 joules to about 150 joules per square centimeter.

17. The method of claim 13, wherein the duration of the short wavelength of laser light is in the range from about 0.45 milliseconds to about 25 milliseconds.

18. The method of claim 14, wherein the duration of longer wavelength of laser light is in the range from about 0.25 milliseconds to about 300 milliseconds.

19. The method of claim 1, wherein step (a) occurs prior to step (b).

20. The method of claim 1, wherein step (a) occurs contemporaneously with step (b).

21. The method of claim 1, wherein the disorder is Nevus of Ota or Nevus of Ito.

22. The method of claim 1 or 21, wherein at least one lesion disposed within the preselected region comprises hypermelanotic color, and wherein applying energy in step (b) lightens the hypernelanotic color of at least one lesion.

23. The method of claim 1 or 21, wherein applying energy in step (b) reduces density of the lesions disposed within the preselected region.

24. The method of claim 1, wherein the disorder is a freckle of Hori.

25. The method of claim 24, wherein in step (b) the energy is applied with a laser light that comprises a short wavelength and a longer wavelength, and wherein the wavelengths are applied sequentially.

26. The method of claim 25, wherein the short wavelength is applied prior to the application of the longer wavelength.

27. The method of claim 25, wherein the short wavelength is applied contemporaneously with the longer wavelength.

28. The method of claim 25, wherein the short wavelength is from about 0.5 to about 0.6 microns.

29. The method of claim 25, wherein the longer wavelength is from about 1.0 to about 1.5 microns.

30. The method of claim 28, wherein the short wavelength of laser light has a fluence in the range of about 2 joules to about 25 joules per square centimeter.

31. The method of claim 29, wherein the longer wavelength of laser light has a fluence in the range of about 4 joules to about 150 joules per square centimeter.

32. The method of claim 28, wherein the laser light duration is from about 0.45 milliseconds to about 40 milliseconds.

33. The method of claim 29, wherein the laser light duration is from about 0.25 milliseconds to about 300 milliseconds.

34. The method of claim 24 or 25, wherein the treatment is repeated weekly, biweekly or monthly until the lesion is reduced or disappears.

35. The method of claim 24, wherein in step (b) the energy is applied with a laser light comprising a short wavelength, a longer wavelength and a longest wavelength, and wherein the wavelengths are applied sequentially.

36. The method of claim 35, wherein the short wavelength is applied, prior to the application of the longer and the longest wavelengths.

37. The method of claim 35, wherein the longest wavelength is applied first, the longer wavelength is applied second, and the short wavelength is applied third.

38. The method of claim 35, wherein the sequential application of more than two wavelengths is random.

39. The method of claim 35, wherein the wavelengths are applied contemporaneously.

40. The method of claim 35, wherein the short wavelength is from about 0.5 to about 0.6 microns.

41. The method of claim 35, wherein the longer wavelength is from about 0.6 to about 1.1 microns.

42. The method of claim 35, wherein the longest wavelength is from about 1.1 to about 1.5 microns.

43. The method of claim 40, wherein the short wavelength of the laser has a fluence in the range from about 2 joules to about 25 joules per square centimeter.

44. The method of claim 41, wherein the longer wavelength of the laser has a fluence in the range from about 4 joules to about 150 joules per square centimeter.

45. The method of claim 42, wherein the longest wavelength of the laser has a fluence in the range from about 4 joules to about 20 joules per square centimeter.

46. The method of claim 43, where in the laser light duration is from about 0.45 milliseconds to about 100 milliseconds.

47. The method of claim 44 or 45, where in the laser light duration is from about 0.25 milliseconds to about 300 milliseconds.

48. The method of claim 35, wherein the treatment is repeated weekly, biweekly or monthly until the lesion is reduced or disappears.

49. The method of claim 24, wherein in step (b) the energy is provided by incoherent lights or intense pulse lights composed of shorter and longer wavelengths.

50. The method of claim 1, wherein the disorder is Nevus of Ota.

51. The method of claim 1, 24, or 50, used in combination with other methods that alleviate the disorder.

52. The method of claim 51, wherein the method that can alleviate the disorder is a use of a Q-switch laser selected from the group consisting: Alexandrite, Ruby, and Nd/YAG.

53. The method of claim 24, wherein at least one lesion disposed within the preselected region comprises hypermelanotic color, and wherein applying energy in step (b) lightens the hypermelanotic color of at least one lesion.

54. The method of claim 24, wherein applying energy in step (b) reduces density of the lesions disposed within the preselected region.
